(19) United States
(12) Patent Application Publication
(10) Pub. No.: US 2024/0350028 A1
(43) Pub. Date: Oct. 24, 2024

(54) METHODS AND SYSTEMS FOR DETERMINING INTRACARDIAC IMPEDANCE

(71) Applicant: St. Jude Medical, Cardiology Division, Inc., St. Paul, MN (US)

(72) Inventor: Peter C. Mooers, Brooklyn Park, MN (US)

(21) Appl. No.: 18/639,348

(22) Filed: Apr. 18, 2024

Related U.S. Application Data
(60) Provisional application No. 63/460,471, filed on Apr. 19, 2023.

(51) Int. Cl. A61B 5/06; A61B 5/00

(52) U.S. Cl. CPC A61B 5/063; A61B 5/6859; A61B 5/6869; A61B 5/743; A61B 2560/0276; A61B 2560/0468; A61B 2562/046

(57) ABSTRACT

A method of determining impedance for a plurality of electrodes on a medical device includes applying a first drive signal between a first pair of adjacent electrodes in the plurality of electrodes and applying a second drive signal between a second pair of adjacent electrodes in the plurality of electrodes. Because the pairs of electrodes are overlapping, the first and second pair of adjacent electrodes include a common electrode. The method further includes applying additional drive signals between additional pairs of adjacent electrodes and measuring an impedance for each pair of adjacent electrodes. The measured impedances can be used to determine contact status or tissue proximity, as well as to detect a faulty electrode or a faulty circuit in the plurality of electrodes.

### CROSS-REFERENCE TO RELATED APPLICATIONS

This application claims benefit of priority to U.S. Provisional Patent Application No. 63/460,471, filed on Apr. 19, 2023, the entire disclosure of which is hereby incorporated by reference.

### TECHNICAL FIELD

The present invention relates generally to catheters, and methods and systems of determining intracardiac impedance between electrodes on the catheter.

### BACKGROUND

Catheters are utilized in a number of operations within the human body, including the heart. In many of these applications, whether collecting data from surrounding tissue or administering treatment, it is important to determine whether portions of the catheter—in particular the electrodes collecting data or administering treatment—are in contact with the adjacent tissue. A number of methods are utilized to determine tissue proximity, including for example monitoring electrocardiogram signals (e.g., voltage measured between electrodes) and/or impedance of an electrode. For example, impedance between an intracardiac electrode and tissue may be determined based on an impedance measured between the intracardiac electrode and a surface patch electrode. When the intracardiac electrode is in the blood pool (i.e., not in contact with tissue), the measured impedance will be lower due to the relatively high conductivity of the blood pool as compared with tissue. Conversely, when the intracardiac electrode is in contact with tissue, the measured impedance will be higher due to the relatively low conductivity of tissue as compared with the blood pool.

However, the impedance between the intracardiac electrode and the surface patch electrode may be affected by attributes of the surface patch electrode (i.e., size of the surface patch electrode, brand/type of surface patch electrode), as well as placement of the surface patch electrode on the body of the patient (i.e. variability in patch to skin conductivity and tissue conductivity). These are some of the factors that can complicate determining tissue proximity based on the measured impedance.

It would be beneficial to develop a method and system for more accurately measuring electrode impedance to determine tissue proximity.

### SUMMARY

According to one aspect, a method of measuring impedance for a plurality of electrodes on a medical device includes applying a first drive signal between a first electrode and a second electrode to measure a first impedance value between the first and second electrodes, applying a second drive signal between the second electrode and a third electrode to measure a second impedance value between the second and third electrodes, and applying a third drive signal between the third electrode and a fourth electrode to measure a third impedance value between the third and fourth electrodes. Each drive signal of the first, second and third drive signals can be applied by a separate signal generator.

According to another aspect, a method of detecting a faulty electrode or a faulty circuit for a medical device having a plurality of electrodes on a distal end of the medical device can include applying a first drive signal between a first pair of adjacent electrodes in the plurality of electrodes and applying a second drive signal between a second pair of adjacent electrodes in the plurality of electrodes, the first and second pair of adjacent electrodes including a common electrode. The method further includes applying additional drive signals between additional pairs of adjacent electrodes, measuring an impedance for each pair of adjacent electrodes, and utilizing the measured impedances to detect a faulty electrode or a faulty circuit in the plurality of electrodes.

According to another aspect, a system for use with a medical device configured for insertion within a patient and having a plurality of electrodes on a distal end of the medical device includes a plurality of measurement circuits, each measurement circuit configured to apply a drive signal to a pair of electrodes among the plurality of electrodes and measure a response for the pair of electrodes associated with the drive signal. The system further includes an electronic control unit (ECU) configured to generate an impedance value for each pair of electrodes based on the measured response. One or more of the electrodes in the plurality of electrodes can be part of two measurement circuits such that adjacent measurement circuits have a common electrode.

### BRIEF DESCRIPTION OF THE DRAWINGS

FIG. 1 is a diagrammatic depiction of a system including a medical device for insertion within a patient, the system configured to utilize impedances between electrodes to determine contact status of the one or more electrodes located at a distal end of the medical device, according to some embodiments.

FIG. 2 is a diagrammatic depiction of a distal end of a medical device having a plurality of splines, each spline including a plurality of electrodes organized in a basket-like array, according to some embodiments.

FIG. 3 is a diagrammatic view of components utilized to measure impedance between electrode pairs located on the medical device, according to some embodiments.

FIG. 4 is a diagrammatic view of components of the medical system used for processing the impedance measurements, according to some embodiments.

FIG. 5 is a flowchart illustrating steps utilized to measure impedance for each electrode pairing for a plurality of electrodes on a medical device, according to some embodiments.

FIG. 6 is a flowchart illustrating steps utilized to detect a faulty electrode or a faulty circuit for a medical device having a plurality of electrodes on a medical device, according to some embodiments.

FIG. 7 is a flowchart illustrating how to utilize measured impedance values to detect a fault electrode or a faulty circuit, according to some embodiments.

### DETAILED DESCRIPTION

According to some embodiments, the claimed invention facilitates measuring impedance between overlapping pairs of catheter electrodes without requiring an external patch or surface electrode attached to the skin of the patient (i.e., patchless impedance system). Without an external patch, the design of the present application eliminates external noise from the patch and/or noise from the patch wires, as well as noise along the tissue path between the electrode and the patch. Moreover, the design of the present application facilitates the collection of more impedance data and better accuracy, which results in better determining tissue proximity of the electrodes. More impedance data also allows for easier detection of opens and shorts between electrodes. In some embodiments, the impedance is a bipolar electrode complex impedance (BECI).

In some embodiments, the catheter electrodes are part of a catheter with a circular design or a round basket/balloon. In an exemplary catheter having eight electrodes, the methods and systems of the present application facilitate the collection of eight impedance measurements. The pairing of electrodes can include overlapping pairs such that impedance values can be collected between, for example, electrodes 1 and 2, electrodes 2 and 3, and electrodes 3 and 4. This contrasts with designs in which electrode pairs do not overlap and, for a four-electrode design, pairs are limited to electrodes 1 and 2 and electrodes 3 and 4.

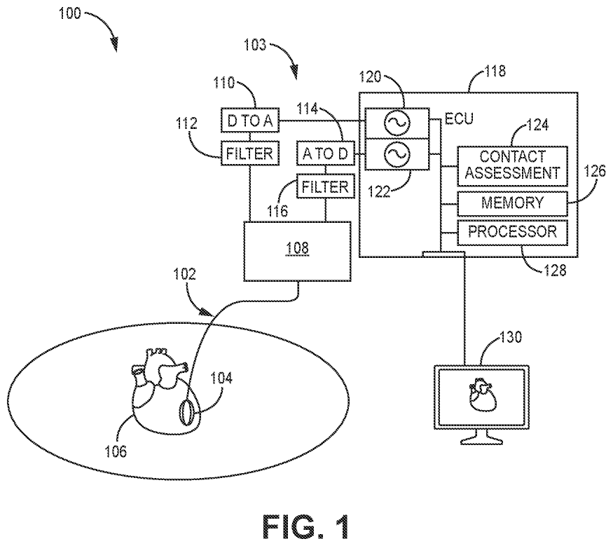

FIG. 1 is a diagrammatic depiction of a system 100 including a medical device 102 and a local system 103. In some embodiments, the local system 103 includes a switch 108, a digital-to-analog (D to A) converter 110, a filter 112, an analog-to-digital (A to D) converter 114, a filter 116, a display 130, and an electronic control unit (ECU) 118 that may include a signal source 120, a synchronous demodulator circuit 122, a contact assessment module 124, a memory 126, and a processor 128.

In some embodiments, the medical device 102 is an elongate medical device, such as a diagnostic and/or therapy catheter, an introducer, sheath, or other similar type of device. The medical device 102 includes a distal end 104 and a proximal end (not shown) that includes a handle operated by a technician as well as interfaces for interfacing the medical device 102 to the local system 103. The distal end 104 may include various sensors and/or components for localization/navigation of the distal end 104 within the patient, mapping of physiological parameters within the patient, and delivery of therapy. In particular, the distal end 104 of the medical device includes a plurality of electrodes that may be utilized for one or more of these purposes for an organ 106 of the patient, such as, for example, the heart.

Contact status of the one or more electrodes located at the distal end 104 of the medical device 102 is determined based on bipolar electrode complex impedance (BECI) measurements. In general, BECI measurements are generated by driving an excitation signal between two electrodes forming a bipolar pair. The resulting voltage at each of the electrodes is measured and utilized to derive a complex impedance signal. Contact assessment module 124 utilizes the measured BECI measurements to determine contact status or tissue proximity.

In the embodiment shown in FIG. 1, signal source 120 is utilized to generate the excitation signal. In some embodiments, signal source 120 generates one or more excitation or drive signals each at a unique frequency. More specifically, the signal generator 120 may generate a plurality of excitation or drive signals having unique frequencies within a range from about 1 kHz to over 500 kHz, more typically within a range of about 2 kHz to 200 kHz, and even more typically between about 10 kHz and about 20 kHz, in one embodiment. The drive signals may each have a constant current, typically in the range of between 1-200 HA, and more typically about 5 μA, in one embodiment. The signal generator 120 may also generate signals involved in, for example, determining a location of the electrodes within the body of the patient, that may be utilized for mapping, navigation, and/or therapy delivery. The digital signal(s) generated by the signal source 120 are converted to analog signal(s) by D-to-A converter 110 and provided via filter 112 and switch 108 to selected bipolar electrodes. In response to the analog signals supplied between selected bipolar electrodes, a resulting voltage is measured at the electrode pairs by the switch 108, the filter 116, the A-to-D converter 114, a synchronous demodulator circuit 122. In some embodiments, switch 108 selects the electrode(s) to monitor in response to the excitation or drive signal delivered. The filter 116 and the A-to-D converter 114 convert the analog signal to a digital signal that can be operated on by the ECU 118. The synchronous demodulator circuit 122 isolates signals from one another based on the frequency of the excitation or drive signal, allowing a plurality of bipolar electrode pairs to be analyzed approximately simultaneously based on the plurality of excitation or drive signals supplied to the electrode pairs.

In some embodiments, the memory 126 may be configured to store data respective of the medical device 102, the patient, and/or other data (e.g., calibration data). Such data may be known before a medical procedure (medical device specific data, number of catheter electrodes, etc.), or may be determined and stored during a procedure. The memory 126 may also be configured to store instructions that, when executed by the processor 128 and/or a contact assessment module 124, cause the ECU 118 to perform one or more methods, steps, functions, or algorithms described herein. For example, but without limitation, the memory 126 may include data and instructions for determining impedances (e.g., bipolar electrode complex impedance or BECI measurements) respective of one or more electrodes on the medical device 102 and utilizing the impedance measurements to determine a contact status of the one or more electrodes. In some embodiments, the contact assessment module 124 utilizes a processor executing instructions stored on the memory 126, an application specific integrated circuit (ASIC), or other type of processor. The ECU may be connected to the display 130, which may display an output of sensed tissue (e.g., heart), the medical device (not shown) and/or determined contact status of the one or more electrodes of the medical device 102.

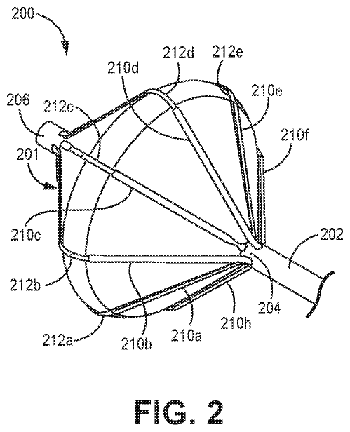

FIG. 2 is an isometric view of a catheter 200. The catheter 200 is an example of a medical device that may be used within the system 100 of FIG. 1. In some embodiments, the catheter 200 is a cardiac catheter. In some embodiments, catheter 200 includes a primary shaft 202, a secondary shaft 206, and a basket assembly 201. In some embodiments, the secondary shaft 206 extends through a primary central shaft lumen of the primary shaft 202. The basket assembly 201 includes a plurality of splines 210a, 210b, 210c, 210d, 210e, 210f, and 210h (collectively splines 210) connected on a proximal end to a distal end 204 of the primary shaft 202 and on a distal end to a distal end of the secondary shaft 206. In the view shown in FIG. 2 spline 210g is not visible. The plurality of splines 210 may be expanded by moving the secondary shaft 206 into the primary shaft 202. In some embodiments, each of the plurality of splines 210 includes at least one corresponding electrode 212a, 212b, 212c, 212d, 212e, 212f, and 212h (collectively electrodes 212). Once again, electrode 212g located on spline 210g is not visible. In other embodiments, the catheter 200 can include additional electrodes not shown in FIG. 2—for example, the catheter 200 could include electrodes that are proximal to and/or distal to the basket assembly 201.

In some embodiments, impedance measurements may be taken between overlapping pairs of adjacent electrodes on the catheter 200—such as, for example, between electrode 212a and 212b, between electrodes 212b and 212c, between electrodes 212c and 212d, etc. Impedance measurements may also be taken between adjacent electrodes 212h and 212a. In this way, two impedance measurements may be generated for each electrode (e.g., with respect to electrode 212b, a first impedance measurement is made between the electrode pair 212a-212b and a second impedance measurement is made between the electrode pair 212b-212c). By overlapping electrode pairs, in an embodiment utilizing eight electrodes (as shown in FIG. 2), eight impedance measurements can be collected for the catheter 200 and those eight measurements can be used to determine contact status or tissue proximity of the electrodes 212. As described below, more impedance data means better accuracy in determining tissue proximity, as well as more data for determining whether the various electrodes 212 are operating properly.

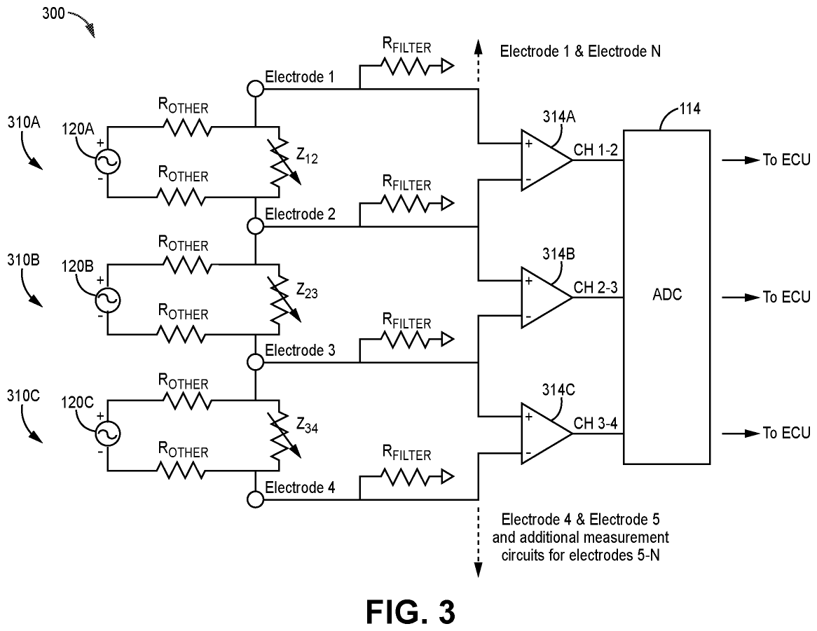

FIG. 3 is a diagrammatic view of circuit elements for measuring impedance between pairs of electrodes 212 of the catheter 200 in FIG. 2, without a surface patch electrode. Three measurement circuits 310A, 310B and 310C are shown in FIG. 3 and each measurement circuit 310A, 310B and 310C includes a pair of electrodes. Electrodes 1, 2, 3 and 4 of FIG. 3 correspond to electrodes 212a, 212b, 212c and 212d, respectively, of the catheter 200 of FIG. 2. Specifically, FIG. 3 shows three electrode pairs-a first pairing between electrodes 1 and 2, a second pairing between electrodes 2 and 3, and a third pairing between electrodes 3 and 4. Additional measurement circuits not shown in FIG. 3 can be used for measuring impedance for the remaining electrode pairs for electrodes 5-8 (212e-212h). Each measurement circuit 310A, 310B and 310C also includes two resistors, labeled as R_{OTHER}. Each of electrodes 1, 2, 3 and 4 also includes a filter, labeled as R_{FILTER}.

The variable impedance Z₁₂ represents the impedance through the blood pool and/or tissue between electrodes 1 and 2. Likewise, the variable impedance Z₂₃ represents the impedance through the blood pool and/or tissue between electrodes 2 and 3 and the variable impedance Z₃₄ represents the impedance through the blood pool and/or tissue between electrodes 3 and 4. The impedance measurements are utilized to determine tissue contact or tissue proximity, i.e. whether the respective electrodes are located in the blood pool (in which case the measured impedance is lower) or adjacent to tissue (in which case the measured impedance increases).

In some embodiments, the measurement circuit 310A is configured to measure impedance between electrodes 1 and 2, and includes a drive signal or signal generator 120A; the measurement circuit 310B is configured to measure impedance between electrodes 2 and 3, and includes a drive signal or signal generator 120B; and the measurement circuit 310C is configured to measure impedance between electrodes 3 and 4, and includes a drive signal or signal generator 120C. Each of the measurement circuits 310A, 310B and 310C includes an operational amplifier 314A, 314B and 314C, respectively.

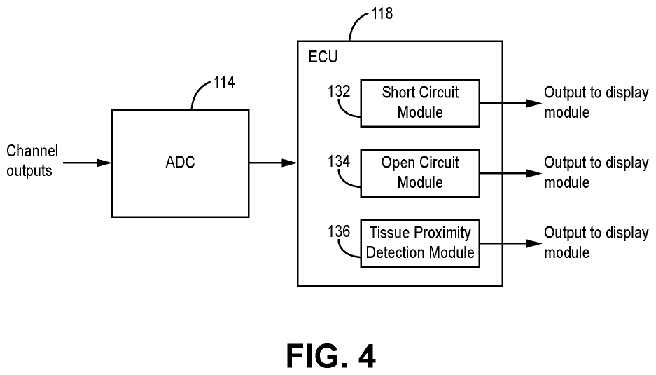

First op-amp 314A includes a first terminal (i.e. positive terminal) connected to electrode 1 and a second terminal (i.e. negative terminal) connected to electrode 2. The output of the op-amp 314A, noted as channel 1-2, reflects the difference in voltage between electrodes 1 and 2. The voltage difference between electrodes 1 and 2 is related to the impedance Z₁₂ between electrodes 1 and 2, which in turn is related to whether the electrodes 1 and 2 are located in the blood pool or are adjacent to tissue. Second op-amp 314B includes a first terminal connected to electrode 2 and a second terminal connected to electrode 3. The output of the op-amp 314B, noted as channel 2-3, reflects the difference in voltage between electrodes 2 and 3. The difference in voltages measured by the op-amp 314B is related to the impedance Z₂₃ between the electrodes 2 and 3. Third op-amp 314C includes a first terminal connected to electrode 3 and a second terminal connected to electrode 4. The output of the op-amp 314C, noted as channel 3-4, reflects the difference in voltage between electrodes 3 and 4. The difference in voltages measured by the op-amp 314C is related to the impedance 734 between the electrodes 3 and 4. The respective outputs of the op-amps 314A, 314B and 314C can be provided to the ADC 114 for digital conversion and provision to the ECU 118 (see FIG. 4) to determine the measured impedance for each electrode pair. Based on the measured impedances, ECU 118 determines the contact status or tissue proximity of the respective electrodes. In some embodiments, the ECU 118 may also utilize the measured impedances to detect fault states of the electrodes (e.g., short-circuits, open circuits, etc.).

Depending on the catheter design, some or all of the electrodes are each part of two different measurement circuits. For example, electrode 2 is paired with electrode 1 in the measurement circuit 310A and with electrode 3 in the measurement circuit 310B. In other words, electrode 2 is a common electrode between the measurement circuits 310A and 310B. As shown in FIG. 3, electrode 4 is paired with electrode 3 in the measurement circuit 310C, but electrode 4 can also be paired with electrode 5 in an additional measurement circuit not shown in FIG. 3. Similarly, electrodes 5 through N can be paired up in additional measurement circuits, with ‘N’ being the total number of electrodes on the catheter.

For the basket design of the catheter 200 of FIG. 2, all eight electrodes can each be part of two different measurement circuits—the last electrode pairing being electrodes 8 and 1 (i.e., electrodes 212h and 212a). Thus, in the example of catheter 200, there are eight electrode pairings and eight impedance measurements—i.e. the number of pairings and impedance measurements is equal to the total number of electrodes. In other embodiments, the first and last electrodes can each be part of only one measurement circuit, whereas the other remaining electrodes are each part of two different measurement circuits, in which case the total number of electrode pairings and impedance measurements is equal to one less than the total number of electrodes.

In some embodiments, each measurement circuit 310A, 310B and 310C includes its own drive signal/signal generator 120A, 120B and 120C, respectively. In some embodiments, the signal generators 120A, 120B and 120C can operate sequentially. In some embodiments, the signal generators 120A, 120B and 120C can operate simultaneously. A single frequency can be used among the measurement circuits 310A, 310B and 310C if the channels are time sliced so there is no overlap of the channel drives. The drive signals can be multiplexed. The drive signals can be intermittent. The drives may need to be blanked (turned off) if used with other measurements, such as magnetic measurements, to avoid interference. In some embodiments, different modulation frequencies can be used for the circuits 310A, 310B and 310C. In some embodiments, the AC signal for generators 120A, 120B and 120C are provided at about 17 KHz, each separated by some given frequency (e.g., 25 Hz). For example, the AC signal for generator 120A is 17.300 KHz, the AC signal for generator 120B is 17.325 KHz, and the AC signal for generator 120C is 17.350 KHz.

In other embodiments, the measurement circuits 310A, 310B and 310C can share a signal generator. A single frequency can be used among the various measurement circuits 310A, 310B and 310C if the channels are time sliced.

As described above, each electrode can be part of two measurement circuits. Similarly, each electrode can be connected to two signal generators. For example, electrode 2 can be commonly connected to two adjacent signal generators-signal generator 120A and signal generator 120B. Thus electrode 2 is the common electrode between the signal generators 120A and 120B. Each electrode can be connected to two op amps. For example, electrode 2 can be commonly connected to two adjacent op amps-op amp 314A and op amp 314B.

Using the methods and systems of the present application, the impedance between electrode pairs is measured directly by the impedance in the blood pool/tissue. Resistive pathways from an external patch and the wires between the electrodes and the patch, all of which contribute to variability, are eliminated in the design of the present application. In a surface patch design, variable resistance and unbalanced loading between electrode pairs can lead to inaccurate impedance measurements. Moreover, little information can be gleaned about non-paired, but adjacent, electrodes in a patch design.

Using the methods and systems of the present application, which include overlapping pairs, more impedance data is collected, which leads to a better determination of tissue proximity. Moreover, the methods and systems of the present application facilitate better detection of faulty electrodes and/or faulty circuits.

FIG. 4 is a schematic of the components of the system 100, including the ADC 114 and ECU 118, which process the channel outputs from the op-amps 314A, 314B and 314C of FIG. 3. The analog output is passed through the ADC 114 to convert the analog output to a digital output, which is then received by the ECU 118. In some embodiments, the ECU 118 includes a short circuit module 132, an open circuit module 134 and a tissue proximity detection module 136.

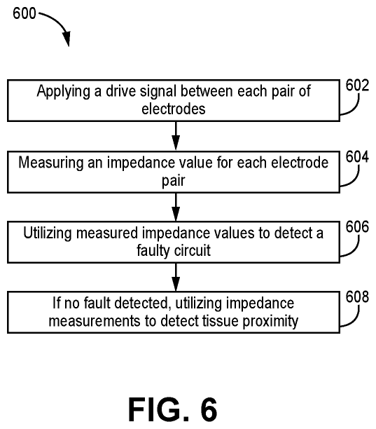

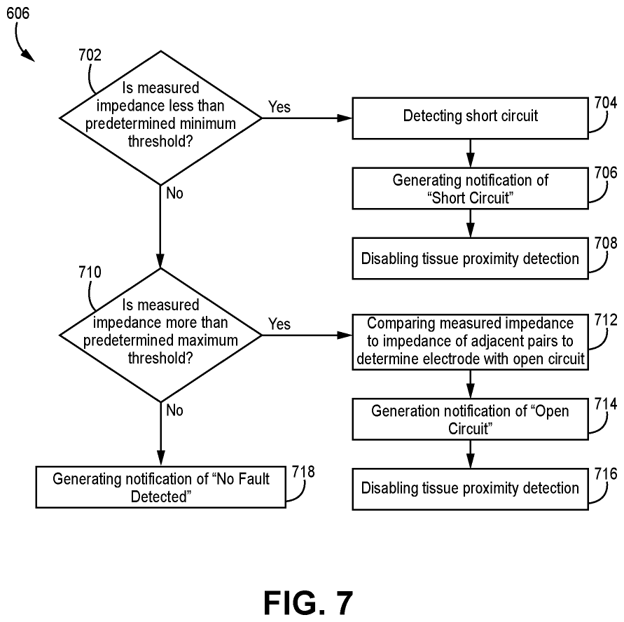

The ECU 118 can use the impedance measurements for each electrode pairing to detect the presence of faulty electrodes or faulty circuits. For example, a short circuit can result from a pair of electrodes physically touching one another, and an open circuit can result if there is a disconnect in the electrical pathway for a particular electrode. If a short circuit is detected by the short circuit module 132, a notification can be generated for the display 130 (see FIG. 1) alerting users of the system 100 of the short. Similarly, if an open circuit is detected by the open circuit module 134, a notification can be generated for the display 130 alerting users of the system 100 of the open circuit. Additional details for detecting a short or open circuit are provided below in reference to FIGS. 6 and 7. Similarly, the ECU 118 can use the impedance measures for each electrode pairing to detect tissue proximity, i.e. whether the electrodes are adjacent to tissue or in the blood pool. The tissue proximity detection module 136 can determine the tissue proximity status and a notification can be generated by the module 136 and relayed to the display 130.

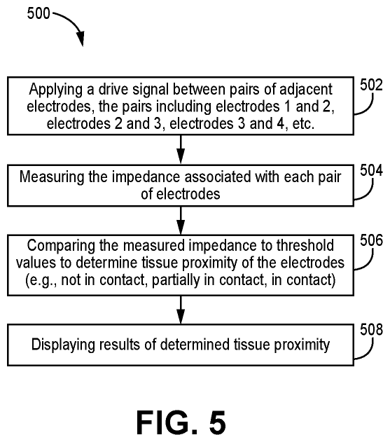

FIG. 5 is a flowchart illustrating steps in a method 500 for measuring impedance for each electrode pairing for a plurality of electrodes on a medical device when the medical device is inside the body of a patient. In some embodiments, the medical device is a catheter, and the electrodes are located on a distal end of the catheter. At step 502, the method 500 includes applying a drive signal between pairs of adjacent electrodes. The electrode pairings overlap such that at least some of the electrodes in the plurality of electrodes are contained within two measurement circuits. In some embodiments, all of the electrodes are contained within two measurement circuits and the number of pairings is equal to the total number of electrodes on the medical device. In some embodiments, the respective drive signals applied at step 502 are applied simultaneously to the plurality of electrodes using frequency division multiplexing (i.e., each drive signal applied at a unique frequency). In other embodiments, the respective drive signals are applied at different times using time division multiplexing.

At step 504, impedance measurements are calculated for each electrode pair. At step 506, the measured impedance is compared to a threshold value to determine tissue proximity of the electrodes in the pairing. In some embodiments, if the measured impedance is below the threshold value, the electrodes are determined to not be in contact with tissue (i.e., in the blood pool), and if the measured impedance is above the threshold value, the electrodes are determined to be in proximity or adjacent to tissue. In some embodiments, the term “contact status” or “tissue proximity” is a binary determination, with the electrode either being “in contact” with the tissue or “not in contact” with the tissue. In other embodiments, tissue proximity may include additional contact states, such as “intermittent contact”. At step 508, the results of the determined tissue proximity are displayed for each electrode pair, such as on the display 130 (FIG. 1).

FIG. 6 is a flowchart illustrating steps in a method 600 utilized to detect a faulty electrode or a faulty circuit for a medical device having a plurality of electrodes. In addition to determining tissue proximity, the measured impedance can also be used for detecting whether the electrodes are functioning properly. At step 602 a drive signal or signals are applied between each of the plurality of electrode pairs. As discussed above, drive signals may be applied simultaneously to each of the plurality of electrode pairs using frequency division multiplexing or drive signals may be applied one at a time (at the same frequency) to each of the plurality of electrode pairs. At step 604, impedance measurements are calculated for each electrode pair. As described above, the pairs of electrodes are overlapping and the number of pairs is equal to either the total number of electrodes on the catheter or one less than the total number of electrodes, depending on the catheter design.

At step 606, the measured impedance values fare utilized to detect one or more fault conditions, including for example short-circuit faults and/or open-circuit faults. In some embodiments, fault detection described in FIG. 6 is continuously analyzed in response to measured impedance values. In some embodiments, fault detection is provided only at start-up/initialization. In some embodiments, a detected fault at one of the plurality of electrode pairs (for example, a short-circuit fault) prevents the measured impedance from being utilized for tissue detection. In some embodiments, fault determinations are made on a pair-by-pair basis. That is, a detected fault associated with the pairing of electrodes 2 and 3 would be utilized to disable tissue proximity detection for that pairing of electrodes 2 and 3, but it would not prevent impedance measurements associated with the pairing of electrodes 1 and 2 or the pairing of electrodes 3 and 4 from being utilized to detect tissue proximity.

At step 608, if no fault is detected in step 606, the impedance measurements are utilized to detect tissue proximity.

FIG. 7 is a flowchart illustrating further analysis under step 606 for determining whether a fault is detected. Steps 702 and 710 in FIG. 7 can be performed for each electrode pairing and its corresponding measured impedance value.

At step 702, the measured impedance is compared to a minimum threshold value. In some embodiments, the minimum threshold value is a predetermined value. In other embodiments, the minimum threshold value may be set or determined based on previous measurements (e.g., baseline measurements). In some embodiments, the minimum threshold is equal to or less than the impedance measured when the electrodes are located in the blood pool. In other embodiments, the minimum threshold is significantly less than the measured or expected impedance between electrodes in the blood pool (e.g., approximately equal to zero Ohms). In general, the impedance measured when the electrodes are in the blood pool represents the lowest impedance state. An impedance less than this value indicates a short-circuit fault condition (typically a result of the electrodes coming into electrical contact with one another).

If at step 702 the measured impedance is less than the minimum threshold value, then at step 704 a short-circuit condition is detected and at step 706 a notification is generated regarding the detected short-circuit. In some embodiments, the notification generated at step 704 identifies both the type of fault detected and the electrodes associated with the fault. For example, if the measured impedance is associated with electrodes 2 and 3, then the notification generated at step 706 would identify both the type of fault detected (e.g., short-circuit) and the electrode pair with which the fault is associated.

At step 708, the tissue proximity detection is disabled for the electrode pair identified as having a short circuit. In other words, the measured impedance for that electrode pair would be discarded for determining tissue proximity or contact status.

If at step 702 the measured impedance value is not less than the minimum threshold, then at step 710 the measured impedance is compared to a threshold maximum value. In some embodiments the threshold maximum value is a predetermined value greater than any expected impedance measured between an electrode pair. If at step 710 the measured impedance is greater than the maximum threshold, then that is indicative of a possible open-circuit fault. In some embodiments, if the measured impedance is greater than a maximum threshold at step 710, then at step 712 the measured impedance associated with adjacent electrode pairs is utilized to determine if a particular electrode is experiencing an open-circuit fault.

If ‘YES’ at step 710, further analysis is done at step 712 to determine which electrode in the electrode pairing has the open circuit. As an example, referring back to FIG. 3, if the measured impedance value for electrodes 2 and 3 (measurement circuit 310B) is more than the maximum threshold, electrode 2 or electrode 3 may have an open circuit. The measured impedance values for the measurement circuit 310A (electrodes 1 and 2) and the measurement circuit 310C (electrodes 3 and 4) can be looked at under step 712. If the impedance value for the measurement circuit 310A is also above the maximum threshold, but the impedance value for the measurement circuit 310C is below the maximum threshold, the common electrode between the two high impedance circuits is electrode 2. In that example, step 712 includes determining an open circuit for electrode 2. Step 714 includes generating a notification that an open circuit is detected. In some embodiments, the notification can specify which electrode on the catheter has the open circuit.

At step 716, the tissue proximity detection is disabled for the electrode identified as having an open circuit and that electrode would be discarded for determining tissue proximity or contact status.

If the answer is ‘NO’ for both decision points 702 and 710 for all electrode pairings, then at step 718, a notification is generated of “No Fault Detected”. In some embodiments, such notification under step 718 (as well as steps 706 and 714) can include a visual alert on the display 130 and/or an audio alert.

By having overlapping electrode pairs (1 and 2; 2 and 3; 3 and 4) compared to distinct pairing (1 and 2; 3 and 4), the methods and systems of the present application facilitate better detection of both short circuits and open circuits. Under a design in which impedance is only measured between electrodes 1 and 2 and between electrodes 3 and 4, a short between electrodes 2 and 3 would not be detected. By contrast, the design of the present application measures impedance of overlapping electrode pairs and thus facilitates detection of a short circuit between electrodes 2 and 3. Similarly, if an open circuit is detected based on a high impedance value for an electrode pairing, the methods and systems of the present application facilitate deciphering which electrode in the electrode pairing has the open circuit.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

### Discussion of Possible Embodiments

The following are non-exclusive descriptions of possible embodiments of the present invention.

According to one aspect, a method of measuring impedance for a plurality of electrodes on a medical device includes applying a first drive signal between a first electrode and a second electrode to measure a first impedance value between the first and second electrodes, applying a second drive signal between the second electrode and a third electrode to measure a second impedance value between the second and third electrodes, and applying a third drive signal between the third electrode and a fourth electrode to measure a third impedance value between the third and fourth electrodes. Each drive signal of the first, second and third drive signals can be applied by a separate signal generator.

The method of the preceding paragraph can optionally include, additionally and/or alternatively any, one or more of the following features, steps, configurations and/or additional components.

In some embodiments, the method can further comprise applying additional drive signals between additional pairs of electrodes, wherein each electrode in the additional pairs of electrodes is connected to two drive signals.

In some embodiments, ‘N’ is equal to a total number of electrodes on the medical device, and an Nth drive signal is applied between the first electrode and the Nth electrode.

In some embodiments, the medical device is a catheter having a plurality of splines, and the plurality of electrodes are arranged on the splines.

In some embodiments, each spline of the plurality of splines includes one or more electrodes.

In some embodiments, the first electrode is located on a first spline of the plurality of splines, the second electrode is located on a second spline of the plurality of splines, the third electrode is located on a third spline of the plurality of splines, and the fourth electrode is located on a fourth spline of the plurality of splines.

In some embodiments, each drive signal of the first, second and third drive signals is applied at a different frequency.

In some embodiments, the method can further comprise detecting a faulty electrode or a faulty circuit in the plurality of electrodes based on the measured impedance values.

In some embodiments, detecting a faulty electrode or a faulty circuit comprises detecting a short circuit for an electrode pair when the measured impedance of the electrode pair is less than a predetermined minimum threshold.

In some embodiments, detecting a faulty electrode or a faulty circuit comprises detecting an open circuit for an electrode pair when the measured impedance of the electrode pair is more than a predetermined maximum threshold.

According to another aspect, a method of detecting a faulty electrode or a faulty circuit for a medical device having a plurality of electrodes on a distal end of the medical device includes applying a first drive signal between a first pair of adjacent electrodes in the plurality of electrodes and applying a second drive signal between a second pair of adjacent electrodes in the plurality of electrodes. The first and second pair of adjacent electrodes can include a common electrode. The method further includes applying additional drive signals between additional pairs of adjacent electrodes, measuring an impedance for each pair of adjacent electrodes, and utilizing the measured impedances to detect a faulty electrode or a faulty circuit in the plurality of electrodes.

The method of the preceding paragraph can optionally include, additionally and/or alternatively any, one or more of the following features, steps, configurations and/or additional components.

In some embodiments, utilizing the measured impedances to detect a faulty electrode or a faulty circuit comprises detecting a short circuit for an electrode pair when the measured impedance of the electrode pair is less than a predetermined minimum threshold.

In some embodiments, utilizing the measured impedances to detect a faulty electrode or a faulty circuit comprises detecting an open circuit for an electrode pair when the measured impedance of the electrode pair is more than a predetermined maximum threshold.

In some embodiments, the method further includes reviewing measured impedances of adjacent pairs of electrodes to determine which electrode in the electrode pair has the open circuit.

In some embodiments, the method further includes generating a notification of whether a faulty electrode or a faulty circuit was detected in the utilizing step.

According to another aspect, a system for use with a medical device configured for insertion within a patient and having a plurality of electrodes on a distal end of the medical device includes a plurality of measurement circuits, each measurement circuit configured to apply a drive signal to a pair of electrodes among the plurality of electrodes and measure a response for the pair of electrodes associated with the drive signal. The system further includes an electronic control unit (ECU) configured to generate an impedance value for each pair of electrodes based on the measured response. One or more of the electrodes in the plurality of electrodes is part of two measurement circuits such that adjacent measurement circuits have a common electrode.

The system of the preceding paragraph can optionally include, additionally and/or alternatively any, one or more of the following features, steps, configurations and/or additional components.

In some embodiments, all electrodes in the plurality of electrodes are part of two measurement circuits.

In some embodiments, the ECU is configured for detecting at least one of a faulty electrode in the plurality of electrodes and a faulty pairing in the plurality of electrodes.

In some embodiments, the ECU comprises a short circuit module configured for detecting a short circuit between a pair of electrodes in the plurality of electrodes.

In some embodiments, the ECU comprises an open circuit module configured for detecting an open circuit for an electrode in the plurality of electrodes.

1. A method of measuring impedance for a plurality of electrodes on a medical device, the method comprising:
applying a first drive signal between a first electrode and a second electrode to measure a first impedance value between the first and second electrodes;
applying a second drive signal between the second electrode and a third electrode to measure a second impedance value between the second and third electrodes; and
applying a third drive signal between the third electrode and a fourth electrode to measure a third impedance value between the third and fourth electrodes,
wherein each drive signal of the first, second and third drive signals is applied by a separate signal generator.

2. The method of claim 1, further comprising applying additional drive signals between additional pairs of electrodes, wherein each electrode in the additional pairs of electrodes is connected to two drive signals.

3. The method of claim 1, wherein ‘N’ is equal to a total number of electrodes on the medical device, and an Nth drive signal is applied between the first electrode and the Nth electrode.

4. The method of claim 1, wherein the medical device is a catheter having a plurality of splines, and the plurality of electrodes are arranged on the splines.

5. The method of claim 4, wherein each spline of the plurality of splines includes one or more electrodes.

6. The method of claim 4, wherein the first electrode is located on a first spline of the plurality of splines, the second electrode is located on a second spline of the plurality of splines, the third electrode is located on a third spline of the plurality of splines, and the fourth electrode is located on a fourth spline of the plurality of splines.

7. The method of claim 1, wherein each drive signal of the first, second and third drive signals is applied at a different frequency.

8. The method of claim 1, further comprising:
detecting a faulty electrode or a faulty circuit in the plurality of electrodes based on the measured impedance values.

9. The method of claim 8, wherein detecting a faulty electrode or a faulty circuit comprises detecting a short circuit for an electrode pair when the measured impedance of the electrode pair is less than a predetermined minimum threshold.

10. The method of claim 8, wherein detecting a faulty electrode or a faulty circuit comprises detecting an open circuit for an electrode pair when the measured impedance of the electrode pair is more than a predetermined maximum threshold.

11. A method of detecting a faulty electrode or a faulty circuit for a medical device having a plurality of electrodes on a distal end of the medical device, the method comprising:
applying a first drive signal between a first pair of adjacent electrodes in the plurality of electrodes;
applying a second drive signal between a second pair of adjacent electrodes in the plurality of electrodes, the first and second pair of adjacent electrodes including a common electrode;
applying additional drive signals between additional pairs of adjacent electrodes;
measuring an impedance for each pair of adjacent electrodes; and
utilizing the measured impedances to detect a faulty electrode or a faulty circuit in the plurality of electrodes.

12. The method of claim 11, wherein utilizing the measured impedances to detect a faulty electrode or a faulty circuit comprises detecting a short circuit for an electrode pair when the measured impedance of the electrode pair is less than a predetermined minimum threshold.

13. The method of claim 11, wherein utilizing the measured impedances to detect a faulty electrode or a faulty circuit comprises detecting an open circuit for an electrode pair when the measured impedance of the electrode pair is more than a predetermined maximum threshold.

14. The method of claim 13, further comprising:
reviewing measured impedances of adjacent pairs of electrodes to determine which electrode in the electrode pair has the open circuit.

15. The method of claim 11, further comprising:
generating a notification of whether a faulty electrode or a faulty circuit was detected in the utilizing step.

16. A system for use with a medical device configured for insertion within a patient and having a plurality of electrodes on a distal end of the medical device, the system comprising:
a plurality of measurement circuits, each measurement circuit configured to apply a drive signal to a pair of electrodes among the plurality of electrodes and measure a response for the pair of electrodes associated with the drive signal; and
an electronic control unit (ECU) configured to generate an impedance value for each pair of electrodes based on the measured response,
wherein one or more of the electrodes in the plurality of electrodes is part of two measurement circuits such that adjacent measurement circuits have a common electrode.

17. The system of claim 16, wherein all electrodes in the plurality of electrodes are part of two measurement circuits.

18. The system of claim 16, wherein the ECU is configured for detecting at least one of a faulty electrode in the plurality of electrodes and a faulty pairing in the plurality of electrodes.

19. The system of claim 18, wherein the ECU comprises a short circuit module configured for detecting a short circuit between a pair of electrodes in the plurality of electrodes.

20. The system of claim 18, wherein the ECU comprises an open circuit module configured for detecting an open circuit for an electrode in the plurality of electrodes.
